(19) United States
(12) Plant Patent Application Publication
(10) Pub. No.: US 2008/0209606 P1
(43) Pub. Date: Aug. 28, 2008

(54) Argyranthemum plant named 'Danargy20'

(75) Inventor: Gabriel Danziger, Moshav Nir-Zvi, IL (US)

Correspondence Address: Richard C. Peet;Foley & Lardner LLP, Washington Harbour, 3000 K Street NW, Suite 500, Washington, DC, 20007-5143, US

(73) Assignee: DANZIGER "DAN" FLOWER FARM

(21) Appl. No.: 11/711,779

(22) Filed: Feb. 28, 2007

(51) Int. Cl. A01H 5/00

(52) U.S. Cl. PLT/406

(57) ABSTRACT

A new and distinct Argyranthemum plant named ‘DANARGY20’ particularly characterized by upright growth habit; single flowers; about 2 to 3 flowers per stem; flowers yellow in color with a distinct brown eye; smooth textured leaves; and early flowering.

### LATIN NAME OF THE GENUS AND SPECIES OF THE CLAIMED PLANT

Argyranthemum frutescens

### VARIETY DENOMINATION

‘DANARGY20’

### BACKGROUND OF THE INVENTION

The present invention relates to a new and distinct cultivar of Argyranthemum plant, botanically known as Argyranthemum frutescens, hereinafter referred to by the cultivar name ‘DANARGY20’.

Argyranthemum, of the Compositae family, consists of annuals, which are grown in most climate zones in the U.S., and only in zones 9 and 10 if it is a perennial plant.

The new Argyranthemum cultivar is a product of a planned breeding program conducted by the inventor, Gabriel Danziger, in Moshav Mishmar Hashiva, Israel. The objective of the breeding program is to develop a new Argyranthemum variety with single, large in diameter flowers, yellow in color with a brown eye, and which are upright on above average length stems.

The new Argyranthemum cultivar originated from a cross-made in a controlled breeding program by the inventor in 2003 in Moshav Mishmar Hashiva, Israel. Open pollination of the seed parent, an unpatented Argyranthemum cultivar designated ‘AR-2-15’, took place in the controlled breeding program in Moshav Mishmar Hashiva, Israel. The new Argyranthemum ‘DANARGY20’ was discovered and selected by the inventor, Gabriel Danziger, in January of 2004 as a single flowering plant within the progeny of the stated cross in a controlled environment in Moshav Mishmar Hashiva, Israel

Asexual reproduction of the new Argyranthemum cultivar by vegetative cuttings was first performed in January of 2004 in Moshav Mishmar Hashiva, Israel, and has demonstrated that the combination of characteristics as herein disclosed for the new cultivar are firmly fixed and retained through successive generations of asexual reproduction. The new cultivar reproduces true to type.

### BRIEF SUMMARY OF THE INVENTION

The following traits have been repeatedly observed and are determined to be unique characteristics of ‘DANARGY20’, which in combination distinguish this Argyranthemum as a new and distinct cultivar:

1. Upright growth habit;

2. Single flowers;

3. About 2 to 3 flowers per stem;

4. Flowers yellow in color with a distinct brown eye;

5. Smooth textured leaves; and

6. Early flowering.

Plants of the new Argyranthemum ‘DANARGY20’ differ from plants of the unpatented, parental cultivar, Argyranthemum ‘AR-2-15’, in the characteristics described in Table 1.

TABLE 1

| | | Parental Cultivar |
|---|---|---|
| | New Cultivar | ‘AR-2-15’ |
| Characteristic | ‘DANARGY20’ | (unpatented) |
| Flower color | Yellow with brown eye | Pink with yellow eye |
| Flower type | Single | Anemone |

Of the many commercial cultivars known to the present inventor, the most similar in comparison to the new Argyranthemum ‘DANARGY20’ is the unpatented, comparison cultivar, Argyranthemum ‘BUTTERFLY’, in the characteristics described in Table 2:

TABLE 2

| | | Comparison Cultivar |
|---|---|---|
| | New Cultivar | ‘BUTTERFLY’ |
| Characteristic | ‘DANARGY20’ | (unpatented) |
| Plant spread | About 50 cm | About 36 cm |
| Flower color | Yellow with brown eye | Yellow with yellow eye |
| Scented Foliage | No | Yes |

### BRIEF DESCRIPTION OF THE DRAWINGS

The accompanying photographic drawings illustrate the overall appearance of the new Argyranthemum ‘DANARGY20’ showing the colors as true as is reasonably possible with colored reproductions of this type. Colors in the photographs may differ slightly from the color value cited in the detailed botanical description, which accurately describe the color of ‘DANARGY20’.

FIG. 1 shows a top view perspective of a typical flowering plant of ‘DANARGY20’ in a 12 cm pot, at 11 weeks of age.

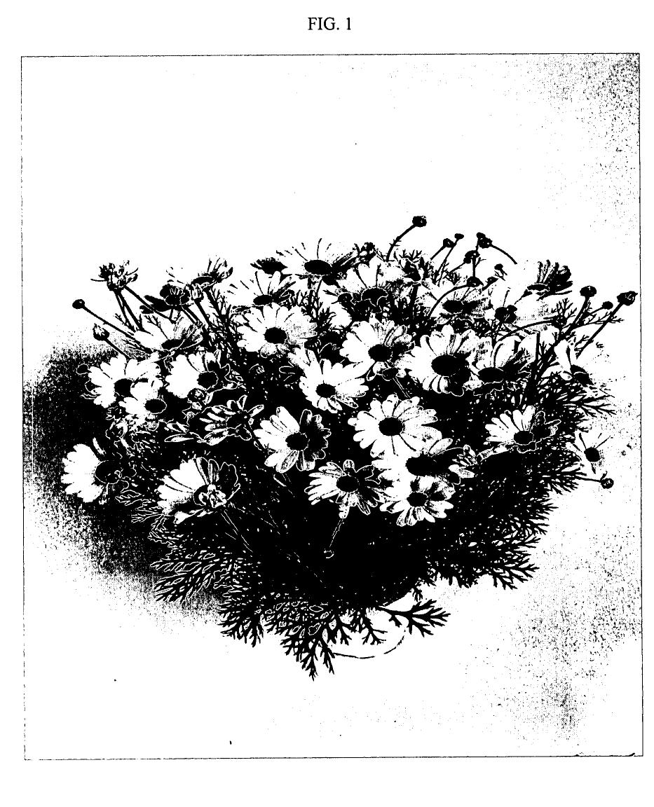

FIG. 2 shows a close-up view of the typical flowers and buds of ‘DANARGY20’ at 11 weeks of age.

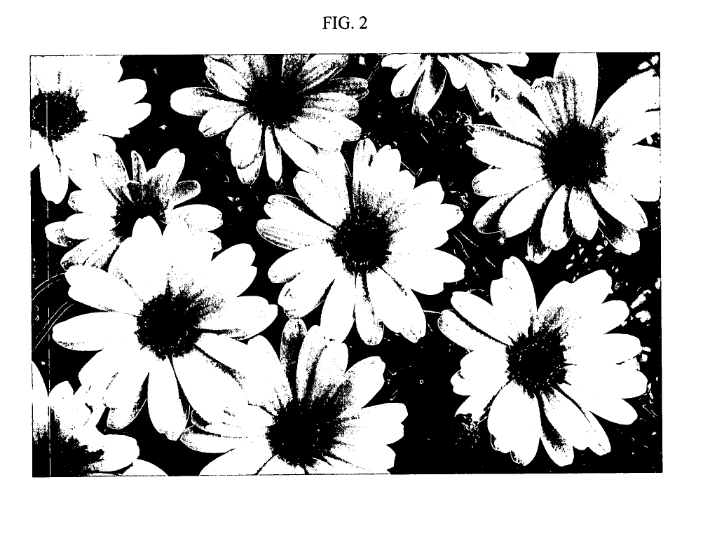

### DETAILED BOTANICAL DESCRIPTION

The new Argyranthemum ‘DANARGY20’ has not been observed under all possible environmental conditions. The phenotype of the new cultivar may vary with variations in environment such as temperature, light intensity, and day length without any change in the genotype of the plant.

The aforementioned photographs, together with the following observations, measurements and values describe the new Argyranthemum ‘DANARGY20’ as grown in a greenhouse in Moshav Mishmar Hashiva, Israel, under conditions which closely approximate those generally used in commercial practice.

Color references are made to The Royal Horticultural Society Colour Chart (RHS), 2001 edition, except where general colors of ordinary significance are used. Color values were taken under daylight conditions at approximately 12:00 noon in Moshav Mishmar Hashiva, Israel. The age of the plant described is 3 months.
Classification:
   Botanical.—Argyranthemum frutescens.
Parentage:
   Seed parent.—Argyranthemum cultivar designated ‘AR-2-15’ (unpatented).
Optimal growth conditions:
   Light intensities.—Full sunlight or slight shade.
   Temperature.—Day: 16° C. to 23° C. Night: 8° C. to 12° C.
   Temperature tolerance.—Tolerant to a low temperature of about 5° C. and tolerant to a high temperature of about 27° C.
   Fertilization.—A balanced fertilizer with level of 100 ppm Nitrogen.
   Growth regulators.—Unnecessary.
Propagation:
   Type.—Tip cuttings of side shoots.
   Rooting habit and description.—Easy to root.
   Time to initiate roots.—About 7 to 10 days at 20° C.
   Time to produce a rooted cutting.—About 14 days at 20° C.
Plant:
General appearance and form:
   Height.—About 40 cm.
   Spread.—About 50 cm.
Shape: Upright growth habit.
Winter hardiness: Hardy in moderate climate.
Vigor: Strong.
Strength: Sturdy upright stems
Crop time: About 7 to 8 weeks for a flowering pot.
Fragrance: None.

Lateral branches:

TABLE 3

| Branch characteristic | Primary | Secondary |
|---|---|---|
| Length | About 20 cm | About 20 cm |
| Diameter | About 4 mm | About 3 mm |
| Internode Length | About 4 cm. | About 3 cm. |
| Texture | Smooth | Smooth |
| Pubescence | None | None |
| Color | RHS 137C, green | RHS 137C, green |

Foliage:
Arrangement and attachment: Alternate
Leaf length: About 7.5 cm.
Leaf width: About 3.5 cm.
Overall shape of leaf: Pinnatipartite.
Apex shape: Rounded.
Base shape: Wedge-shaped.
Margin: Serrate.
Texture (both surfaces): Slightly warty.
Pubescence (both surfaces): None.
Color of upper surface:
   Mature leaf.—RHS 137B, green.
   Immature leaf.—RHS 137B, green.
Color of lower surface:
   Mature leaf.—RHS 137C, green.
   Immature leaf.—RHS 137C, green.
Venation:
   Pattern.—Parallel.
   Color.—Upper surface: RHS 137C, green. Lower surface: RHS 137D, green.
Petiole:
   Length.—About 20 mm.
   Width.—About 2.5 mm.
   Color.—RHS 137D, green.
Leaf fragrance: None.
Flower description:
Natural flowering season: All year round in moderate climate. Flowering occurs between November and March outdoors in Israel.
Flowering response time: About 7 weeks from planting.
Flowering longevity: On the plant: Flowering of a single flower lasts for 20 days.
Flower fragrance: None.
Quantity of flowers and buds per plant: Approximately 150 to 200.
Flower bud (about 15 to 20 days prior to opening):
   Shape.—Round.
   Length.—About 4 to 5 mm.
   Diameter.—About 5 to 7 mm.
   Texture.—Rough.
   Color.—RHS 199B, grey-brown.
Flower type: Solitary.
Flower form: Cruciform.
Flower symmetry: Symmetric.
Orientation at opening: Slanted upward.
Corolla size:
   Diameter (spread).—About 10 mm.
   Overall flower height.—About 15 to 20 mm.
   Length of corolla tube.—About 6 mm.
   Diameter of corolla tube.—About 15 mm.
Petals:
   Quantity.—About 20 to 23.
   Length.—About 25 mm.
   Width.—About 8 mm.
   Overall shape.—Oblong and gameopetalous.
   Apex.—Rectuse.
   Base.—Attenuate.
   Margin.—Entire.
   Texture (upper & lower surfaces).—Smooth.
   Color.—Upper surface, when opening: RHS 2A, yellow. Upper surface, opened flower: RHS 2C, yellow. Lower surface, when opening: RHS 2D, yellow. Lower surface, opened flower: RHS 2D, yellow.
Calyx:
   Sepals.—Attached to the receptacle bottom Quantity: About 15 to 17. Length: About 2 to 3 mm. Width: About 1.0 mm. Overall shape: Oval. Apex: Rounded. Base: Obtuse. Margin: Entire. Texture: Upper surface: Smooth Lower surface: Smooth. Upper surface color: RHS 144C, yellow green. Lower surface color: RHS 144B, yellow green. Anthocyanin pigmentation of calyx limb: Absent.
   Pedicel.—Aspect: Upright. Length: About 65 mm. Diameter: About 1.0 mm. Angle: About 80° from vertical. Texture: Smooth. Color: RHS 146B, yellow green.
Reproductive organs:
Androecium:
   Stamen.—Number: 5 (fused as tube). Arrangement: Didynamous. Type: Basifixed. Dehiscence of stamens: Extrorse. Color: RHS 13A, yellow.
   Anthers.—Shape: Rounded. Length: About 0.5 mm. Color: RHS 13A, yellow.
   Filaments.—Length: About 5 mm. Color: RHS 145C, yellow green.
   Pollen.—Amount: Abundant Color: RHS 13A, yellow. Pollination requirements: None.
Gynoecium:
   Pistils.—Quantity: About 200. Length: About 5 mm.
   Stigma.—Shape: Stringy. Width: About 2 mm. Color: RHS 12B, yellow.
   Style.—Shape: Terete. Length: About 2 mm.
   Ovary.—Shape: Ellipsoid. Position: Superior. Length: About 1.5 mm. Width: About 0.7 mm. Color: RHS 149D, yellow green.
Seeds:
   Quantity per fruit.—About 1.
   Shape.—Oblong.
   Length.—About 2 mm.
   Width.—About 1 mm.
   Texture.—Smooth.
   Color.—RHS 161A, gray-yellow.
Fruit:
   Type.—Achene.
   Shape.—Oblong.
   Length.—About 2 mm.
   Width.—About 1 mm.
   Texture.—Smooth.
   Color.—RHS 161A, gray-yellow.
Disease/pest resistance: Not tested.
Disease/pest susceptibility: No susceptibility was observed under regular growing conditions.

I claim:

1. A new and distinct Argyranthemum plant named ‘DANARGY20’, as illustrated and described herein.
